(19) United States
(12) Patent Application Publication
(10) Pub. No.: US 2004/0024466 A1
(43) Pub. Date: Feb. 5, 2004

(54) Jaw transplant consisting of natural bone material

(76) Inventors: Klaus Heerklotz, Zirndorf (DE); Karl Koschatzky, Erlangen (DE); Manfred Kruger, Arnoldshain (DE); Bernd Fohlinger, Erlangen (DE)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/276,240

(22) PCT Filed: Apr. 9, 2001
(86) PCT No.: PCT/EP01/04053

(30) Foreign Application Priority Data
May 26, 2000 (DE) 100 26 306.2

(51) Int. Cl. A61F 2/28

(52) U.S. Cl. 623/17.17; 623/23.63; 623/919

(57) ABSTRACT

A bone transplant consists of a transplant body which is curved in a channel shape and which consists of bone material of human or animal origin.

The present invention relates to a transplant for the anatomical restoration of the bone shape of a defective or atrophied jawbone ridge (onlay sandwich augmentation).

The success rate of enossal transplant methods has been sufficiently scientifically demonstrated. Implantology is today considered a clinically established treatment measure with a sufficient local bone availability, with a strict indication, a careful surgical technique and an exact prosthetic fitting.

It is the object of the present invention to provide an improved jawbone transplant which accelerates the healing process in the patient.

This object is satisfied by the features of claim 1.

The transplant in accordance with the invention is matched to the anatomical shape of the jawbone due to its arched formation in a channel-like manner, on the one hand. Bone replacement material (bone powder or bone chips) introduced into the defect for embedding can be held locally hereby and the augmentation of missing bone can moreover be brought about, on the other hand.

The transplant further serves as a stable cover of the defective location filled with ground bone material and thus prevents migration of the bone replacement material. The transplant in accordance with the invention does not damage the transplant bed and does not influence imaging processes, or does so only slightly. The anatomical shape of a defective or atrophied jawbone ridge and/or of its side walls can be restored by the transplant in accordance with the invention.

A special advantage of the transplant in accordance with the invention is provided by the material used which does not represent a foreign body due to its biological origin. The transplant manufactured from bone material thereby contributes to the fixing and fusion between the transplant and the transplant bed in that it is converted into the body's own tissue during the healing.

The transplant in accordance with the invention can be used both in the whole upper jaw and in the whole lower jaw for augmentative measures in the different shapes and for a horizontal and vertical gaining of bone (jaw ridge widening and jaw ride raising), for the later or simultaneous implantation of a metallic prosthesis and for the restoring of bone defects in general.

Further advantageous embodiments of the jawbone transplant in accordance with the present invention are set forth in the description, in the drawing and in the dependent claims.

In accordance with a first advantageous embodiment of the invention, the transplant body can have one or more openings, through bores or slits. Such openings can serve for the application of a fixing by pins, screws or nails, on the one hand. Furthermore, such openings also accelerate biological conversion and they can be used for the later introduction of a metallic implant for prosthetic fitting.

The transplant body can extend substantially in a straight line in the longitudinal direction or it can be made curved for a better matching to the anatomy of the jawbone in the longitudinal direction.

It is ensured by the arched formation of the transplant body in a channel-like manner that a matching to the anatomy of the jawbone can take place, on the one hand. The jawbone transplant can be used to hold locally bone replacement material introduced into the defect site, on the other hand. In accordance with a further embodiment of the invention, the transplant body is made substantially in a U shape in cross-section for this purpose so that it has two parallel side walls, or side walls which extend approximately in parallel, which form an extension of the jaw wall on the cheek side or of the cheek wall on the tongue side, whereby the jawbone ridge can be built over in the manner of a tunnel with the help of the transplant.

The geometry of the transplant body made arched in a channel-like manner can differ in dependence on the demand. The surfaces of the transplant body can extend—in a mathematical sense—continuously or discontinuously, i.e. the tunnel arch formed by the transplant body can be made curved, continuously arched, but also of part pieces which adjoin one another in a discontinuous manner such that the outer contour and/or the inner contour of the transplant body forms a progression considered in cross-section. The two limbs of a transplant body substantially made in U shape can be connected to one another in one piece by a connection section which—considered in cross-section—is made planar or as a part circle (e.g. a quarter circle or semi-circle), whereby the angle enclosed by the limbs of the transplant body together are predetermined. In the case of a connection section of quarter circle shape, a transplant body made in a channel-like arched manner still results accordingly, but is made more in a V shape considered in cross-section.

The jawbone transplant in accordance with the invention can, as described above, have wall sections which extend parallel to one another or which stand at an angle to one another. These wall sections can have the same height or be of different height so that an approximately L-shaped cross section results.

In accordance with a preferred embodiment of the invention, the material of the transplant body is composed of preserved and sterile bone material of human origin or of animal origin, in particular of preserved and sterile bone material of bovine, porcine or equine origin.

In accordance with the invention, the material can be composed of processed, preserved and sterile bone material of human origin, a so-called allograft, or of processed, preserved and sterile bone material of animal origin, a so-called xenograft. Furthermore, the bone material can be composed of spongeous, cortical or compact bone or of composites resulting therefrom and, optionally, be charged with bone growth factors (BMPs). The use of fully or partly demineralized bone is also possible.

In accordance with the present invention, a suitable allogenic or xenogenic bone material can be processed such that it is preserved, storable and sterile and can be used for the intended purpose. The preservation of the bone material can take place, for example, by means of freeze drying. However, the bone material is preferably produced by solvent dehydration of collagenic bone material by means of an organic solvent miscible with water, e.g. methanol, propanol, isopropanol, acetone methyl ethyl ketone or mixtures of these solvents. The preservation and sterilization of the bone material in accordance with these processes is also the subject of the patent DE 29 06 650 whose content is taken up into the disclosure of the present application by this reference.

This process serves for the manufacture of preserved transplants and allows a dehydration and exposure right down to the fine structure of the material such that the processed bone material has a very similar structure to the natural bone in the histological image and the desired properties of the starting material are thus maintained. This process of solvent dehydration furthermore has the advantage that a substantially lower apparatus effort is required in comparison with freeze drying.

The bone material can furthermore also be produced by solvent dehydration of bone material containing collagen with subsequent terminal sterilization, in particular by radiation with gamma rays or electron beams, but also by ethylene oxide or thermal processes.

The bone material can alternatively be produced by aseptic processing of bone material containing collagen without terminal sterilization, with a complete or partial demineralization also being possible. The demineralization of the bone material in accordance with this process is also the subject of the German patent application 19849984.1 whose content is taken up into the disclosure of the present application by this reference.

A further possibility lies in the fact of charging the preserved bone material with bone growth factors (BMPs) to accelerate the healing process.

The transplant can be matched in its size to the defect to be covered, which takes place by sculpturing standardized shapes of the transplant.

In the region of the frontal jaw zone, a transplant body having a width of 8 mm can be sufficient. Otherwise, the outer dimensions differ according to place of use and amount to, for example: length (L) approximately 8 up to 22 mm; width (B) approximately 7 up to 8 mm; wall thickness (W) approximately 1 up to 2 mm; height (H) approximately 7 up to 10 mm.

For stabilization and adaptation, the transplant shaped from bone material can be fixed in the body's own present bone by means of screws, pins or nails (“onlay sandwich augmentation”). A membrane should subsequently be placed over the transplant since a tight wound closure is an important requirement for a complication free healing of the augmentation.

The time profile of the healing depends on the reactivity of the bed and takes weeks up to months. As a rule, healing is so advanced after 6 to 8 months that, for example, a transplant insertion can be carried out in the newly built up bone bed. The healing can be inspected by means of X-rays, biopsy, CT or other diagnostic methods.

The present invention will be described purely by way of example in the following with reference to embodiments and to the enclosed drawings.

There are shown:

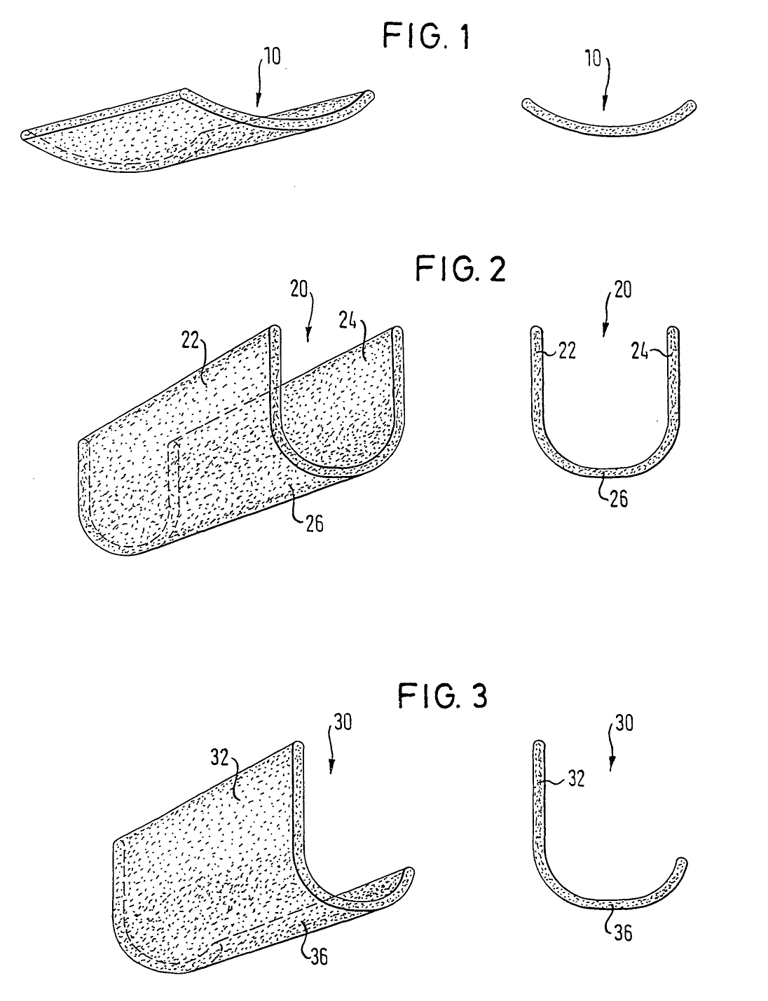

FIG. 1 a perspective view and a cross-sectional view of a first embodiment of a jawbone transplant;

FIG. 2 a perspective view and a cross-sectional view of a second embodiment of a jawbone transplant;

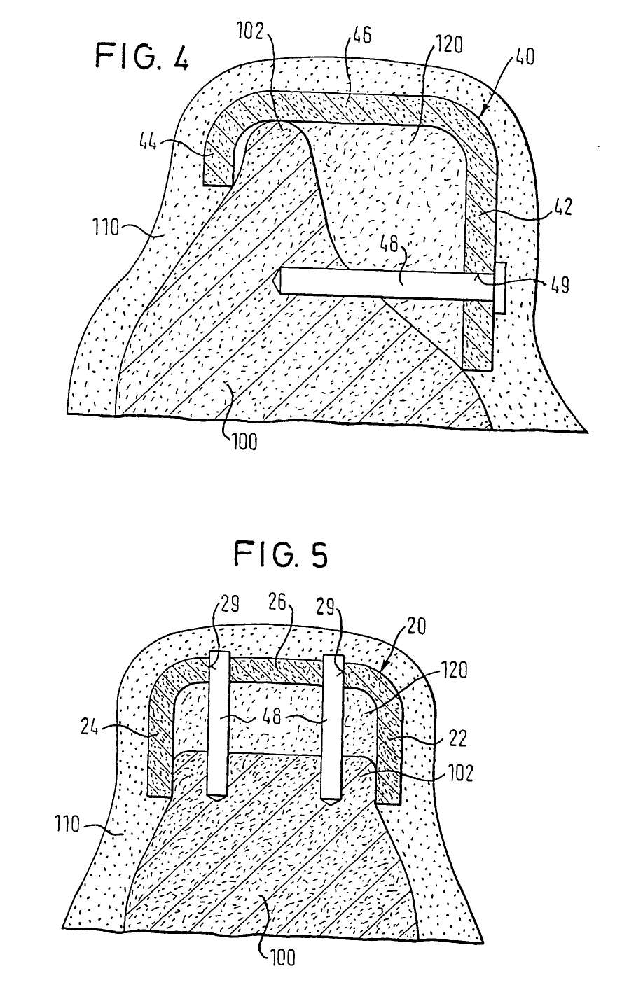

FIG. 3 a perspective view and a cross-sectional view of a third embodiment of a jawbone transplant;

FIG. 4 a cross-sectional view of an atrophied jawbone ridge with an augmentative measure;

FIG. 5 a cross-sectional view of a jawbone ridge raised by augmentation;

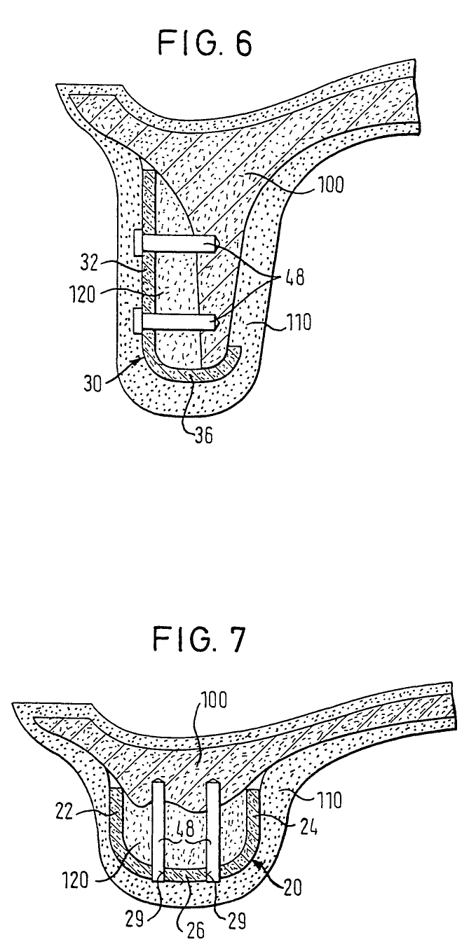

FIG. 6 a cross-sectional view of an atrophied jaw with an augmentative measure; and

FIG. 7 a cross-sectional view of a jaw with an augmentative measure.

The jawbone implant shown in FIG. 1 consists of a transplant body 10 which is made in an arched channel-like manner and which is produced in one piece from spongeous, cortical or compact bone material of human origin or of animal origin. The transplant body forms a shell which is made in a straight line in the longitudinal direction and with a channel-like curve transversely to the longitudinal direction. The transplant body 10 is made in a rectangular manner in a plan view.

FIG. 2 shows a further embodiment of a transplant body 20 which is made of the same bone material as the transplant body 10 and which is likewise made with a channel-like arch. It can be recognized in the cross-sectional view of FIG. 2 that the transplant body 20 has the shape of a U in cross-section, with the limbs of the U forming side walls 22, 24 which extend substantially parallel to one another and which are connected to one another by an arched channel section 26. In this embodiment, the two wall sections 22 and 24 have the same vertical extent.

FIG. 3 shows a third embodiment of a jawbone transplant which consists of a transplant body 30 which is made in an arched channel-like manner and which substantially corresponds to the embodiment of FIG. 2, but with the right hand wall section 24 shown in FIG. 2 being omitted. In this respect, the transplant body 30 consists only of one wall section 32 which is connected in one piece to a channel section 35 formed in a curved manner.

A cross-sectional view of an atrophied jawbone ridge 102 is shown in FIG. 4 on which an augmentative measure has been carried out. For this purpose, a jawbone transplant 40 in accordance with the invention has been provided which substantially corresponds to the embodiment of FIG. 3, with, however, a further short wall section 44 being provided in addition to a wall section 42 and a channel section 46, said wall section 44 extending approximately parallel to the wall section 42, but having only approximately 30% of its vertical extent. As can be recognized with this embodiment, the base or the roof of the channel section 46 can be made planar, i.e. a continuously curved design is not necessary.

In the arrangement of FIG. 4, the jawbone transplant 40 is additionally fixed in the jawbone 100 with the help of pins 48, with the pins 48 being inserted through bores 49 in the transplant body 46. A fixing of the jawbone transplants can taken place with the help of pins, nails, screws or the like. An additional fixing with a membrane over the transplant can take place to prevent immigration of the soft tissue.

As FIG. 4 shows, that hollow space which is formed by the jawbone 100, or the jawbone ridge 102, and the transplant body 40 is filled with bone replacement material 120 in the form of bone powder or of bone chips.

FIG. 5 shows an application of the jawbone transplant 20 of FIG. 2 to raise the jawbone ridge by augmentation. Here, the jawbone transplant 20, which is U shaped in cross-section, is placed onto a jawbone ridge 102 of a jawbone 100 such that the wall sections 22 and 24 extend the jaw wall on the cheek side and the jaw wall on the tongue side, with a hollow space being formed between the transplant body 20 and the jawbone ridge 102 which is filled with bone replacement material 120. A securing with the help of pins 48 is also provided with the embodiment in addition to the fixing of the transplant body 20 with the help of the mucous membrane 10, said pins 48 being inserted through bores 29 in the channel section 26 of the transplant body 20 and being fixed in the jawbone 100.

FIG. 6 shows an application of the jawbone transplant 30 of FIG. 3 with the enlargement of an atrophied jaw. As can be recognized here, the wall section 32 of the jawbone transplant 30 replicates a jaw wall on the cheek side, with the channel section 36 partly engaging around the lower end of the jawbone 100 still present. In other respects, the same reference numerals designate the same parts in this Figure, as also in the other Figures, so that—to avoid repetition—reference can be made to the description of the preceding Figures.

The augmentation of a jaw is shown in FIG. 7 with the help of the jawbone transplant 20 which—similar to FIG. 5—is fixed in the jawbone 100 with the help of pins 48. An additional fixing of a membrane over the transplant is also possible here to prevent immigration of the soft tissue.

A hollow space, into which bone replacement materials can be introduced and held locally, can be created with the help of the jawbone transplant in accordance with the invention by setting the transplant body onto the jawbone. The channel-like arching required for this can be realized by curved or planar part sections of the transplant body.

Reference numeral list

| | |
|---|---|
| 10, 20, 30 | transplant body |
| 22, 24 | wall section |
| 26 | channel-like section |
| 29 | bores |
| 32 | wall section |
| 36 | channel-like section |
| 40 | transplant body |
| 42 | wall section |
| 44 | wall section |
| 46 | channel-like section |
| 48 | pin |
| 49 | bore |
| 100 | jawbone |
| 102 | jawbone ridge |
| 110 | mucous membrane |
| 120 | bone replacement material |

1. A jawbone transplant consisting of a transplant body (10, 20, 30, 40) made in an arched channel-like manner from spongeous, cortical or compact bone material of human origin or of animal origin.

2. A jawbone transplant in accordance with claim 1, characterized in that the transplant body (10, 20, 30, 40) has one or more openings (29, 49), throughbores or slits.

3. A jawbone transplant in accordance with claim 1, characterized in that the transplant body is made curved in the longitudinal direction.

4. A jawbone transplant in accordance with claim 1, characterized in that the transplant body (20, 30, 40) is made substantially in a U shape in cross-section.

5. A jawbone transplant in accordance with claim 1, characterized in that the transplant body (40) is made substantially in a U shape in cross-section, with the limbs (42, 44) of the U being of different length.

6. A jawbone transplant in accordance with any one of the preceding claims, characterized in that the material of the transplant body (10, 20, 30, 40) consists of preserved and sterile bone material.

7. A jawbone transplant in accordance with any one of the preceding claims, characterized in that the bone material is produced by solvent dehydration of collagenic bone material by means of an organic solvent miscible with water, e.g. by means of methanol, ethanol, propanol, isopropanol, acetone, methyl ethyl ketone or mixtures of these solvents.

8. A jawbone transplant in accordance with any one of the preceding claims, characterized in that the bone material is produced by solvent dehydration of collagenic bone material with subsequent terminal sterilization, in particular by radiation with gamma rays or electron beams.

9. A jawbone transplant in accordance with any one of the preceding claims 1-5, characterized in that the bone material is produced by aseptic processing of collagenic bone material, in particular with complete or partial demineralization, without terminal sterilization.

10. A jawbone transplant in accordance with any one of the preceding claims, characterized in that the bone material is charged with at least one bone growth factor (BMP).
